(19) United States
(12) Patent Application Publication
(10) Pub. No.: US 2011/0020452 A1
(43) Pub. Date: Jan. 27, 2011

(54) PROGENITOR CELL REPLICATION AND DIFFERENTIATION IN 3D

(75) Inventor: Jeremy J. Mao, Closter, NJ (US)

Correspondence Address: SNR DENTON US LLP, P.O. BOX 061080, CHICAGO, IL, 60606-1080, US

(73) Assignee: The Trustees of Columbia University in the City of New York, New York, NY (US)

(21) Appl. No.: 12/438,543

(22) PCT Filed: Aug. 22, 2007
(86) PCT No.: PCT/US07/18586

§ 371 Date: Oct. 8, 2010

Related U.S. Application Data
(60) Provisional application No. 60/823,249, filed on Aug. 22, 2006.

(51) Int. Cl. A61K 9/00; A61K 35/12; A61P 43/00

(52) U.S. Cl. 424/488; 424/484; 424/93.7

(57) ABSTRACT

The present invention is directed to a new approach towards in situ differentiation of tissue progenitor cells, without the coventional requirements of weeks of cellular manipulation and treatment. Such approach circumvents the need for 2D culturing and differentiation of tissue progenitor cells before implanting in a 3D biocompatible matrix, thus providing convenience, cost savings, and time savings. One aspect of the invention provides an engineered tissue composition comprising substantially undifferentiated tissue progenitor cells in a biphasic matrix material, along with growth factors or encapsulated growth factors. Another aspect of the invention includes methods for making the compositions described herein. Another aspect of the invention includes methods of therapeutic treatment using the compositions described herein.

### CROSS-REFERENCE TO RELATED APPLICATIONS

This application claims priority to U.S. Provisional Application Ser. No. 60/823,249, filed Aug. 22, 2006, which is hereby incorporated by reference in its entirety.

### STATEMENT REGARDING FEDERALLY SPONSORED RESEARCH OR DEVELOPMENT

This invention was made in part with U.S. Government support under U.S. National Institutes of Health Grant Nos. R01DE15391 and R01EB02332. The U.S. Government has certain rights in the invention.

### INCORPORATION-BY-REFERENCE OF MATERIAL SUBMITTED ON A COMPACT DISC

Not Applicable.

### FIELD OF THE INVENTION

The present invention generally relates to stem cell differentiation and engineered tissue compositions.

### BACKGROUND

Human mesenchymal stem cells (hMSC) have been shown to differentiate into chondrocytes and form cartilage-like tissues when cultured with TGFβ3. Previous attempts to engineer cartilage using hMSC have depended on in vitro pre-differentiation in 2D flat cell culture plates. Such techniques can substantially increase the time of implant fabrication and can suffer from phenotypic loss upon the withdrawal of chondrogenic medium.

Thus, there exists a need for an engineered tissue construct that can be formed without the need of pre-differentiation of tissue progenitor cells.

### SUMMARY

Disclosed herein is a new approach towards in situ differentiation of tissue progenitor cells, without the coventional requirements of weeks of cellular manipulation and treatment. Such approach circumvents the need for 2D culturing and differentiation of tissue progenitor cells before implanting in a 3D biocompatible matrix, thus providing convenience, cost savings, and time savings.

One aspect of the invention provides a tissue composition comprising a substantially undifferentiated tissue progenitor cell, a tissue growth factor, and a biphasic matrix material. The tissue growth factor can be encapsulated in a controlled release delivery system. The biphasic matrix material has a substantially liquid phase and a gelled phase. And the tissue progenitor cell and the growth factor or encapsulated growth factor are infused in the biphasic matrix material, so forming the engineered tissue composition.

A further aspect of the invention provides a method of forming an engineered tissue composition. Such method includes providing a substantially undifferentiated tissue progenitor cell; a tissue growth factor or a tissue growth factor encapsulated in a controlled release delivery system; and a biphasic matrix material having a substantially liquid phase and a gelled phase. Such method also includes introducing the substantially undifferentiated tissue progenitor cell into the substantially liquid phase of the biphasic matrix material. Such method also includes introducing the tissue growth factor or the encapsulated tissue growth factor into the substantially liquid phase of the biphasic matrix material. Such method also includes forming a substantially liquid phase tissue composition comprising the biphasic matrix material, the tissue progenitor cell, and the tissue growth factor or the encapsulated tissue growth factor. In various configurations, the method of forming the engineered tissue composition can include forming a gelled tissue composition from the substantially liquid phase tissue composition.

Yet another aspect of the invention provides a method of treating a tissue defect comprising introducing a composition of the invention into a subject in need thereof.

A still further aspect of the invention provides a method of treating a tissue defect. Such method includes providing a substantially undifferentiated tissue progenitor cell; a tissue growth factor or a tissue growth factor encapsulated in a controlled release delivery system; and a biphasic matrix material having a substantially liquid phase and a gelled phase. Such method also includes introducing the substantially undifferentiated tissue progenitor cell into the substantially liquid phase of the biphasic matrix material. Such method also includes introducing the tissue growth factor or the encapsulated tissue growth factor into the substantially liquid phase of the biphasic matrix material. Such method also includes introducing a composition comprising the biphasic matrix material, the tissue progenitor cell, and the tissue growth factor or the encapsulated tissue growth factor into a subject in need thereof.

In various embodiments of the methods of treatment, at least a portion of the tissue progenitor cells remain substantially undifferentiated upon introduction of the composition into the subject. In some configurations, a majority of the tissue progenitor cells remain substantially undifferentiated upon introduction of the composition into the subject, while in other configurations, substantially all of the tissue progenitor cells remain substantially undifferentiated upon introduction of the composition into the subject. In various embodiments of the methods of treatment, the substantially undifferentiated tissue progenitor cells differentiate in situ after introduction of the composition into the subject. In various embodiments of the methods of treatment, the subject is a mammalian subject. In some configurations, the subject is a non-human mammalian subject, while in other configurations, the subject is a human.

In various embodiments of the compositions and methods, the biphasic matrix material is a thermosensitive biphasic matrix material. In some configurations, the thermosensitive biphasic matrix material can have a substantially liquid phase at about room temperature. In some configurations, the thermosensitive biphasic matrix material can have a gelled phase at about 37° C. In some configurations, the thermosensitive biphasic matrix material is a chitosan-GP matrix.

In various embodiments of the compositions and methods, the biphasic matrix material is injectable in the substantially liquid phase and is in the gelled phase after injection into a subject. In various embodiments of the methods of treatment, a composition of the invention is injected in a liquid phase into the subject.

In various embodiments of the compositions and methods, the tissue progenitor cell is a mesenchymal stem cell (MSC). In some configurations, the tissue progenitor cell is a human mesenchymal stem cell (hMSC).

In various embodiments of the compositions and methods, the tissue growth factor is a chondrogenic growth factor. In some configurations, the tissue growth factor is TGFβ3.

In various embodiments of the compositions and methods, the controlled release delivery system is a polymeric microsphere. In some configurations, the controlled release delivery system is a poly(DL-lactic-co-glycolic acid (PLGA) polymeric microsphere.

In various embodiments of the compositions and methods, the substantially undifferentiated tissue progenitor cells are is not treated with a growth factor before being introduced into the biphasic matrix material.

Other objects and features will be in part apparent and in part pointed out hereinafter.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 is a schematic diagram of conventional and present approaches for the fabrication of chondrogenic injectable tissue engineered constructs. Bone marrow can be aspirated from the marrow cavity of bones, such as the tibia and iliac crest. In the conventional approach for engineering cartilage, tissue engineers isolate mesenchymal progenitors from the bone marrow using negative selection techniques. Mesenchymal stem cells are then plated, culture expanded, and treated with growth factors to induce chondrogenic differentiation. This laborious process can take up to several weeks of laboratory manipulation. In the present approach, bone marrow progenitors can be directly added to a chondrogenic gel material that includes controlled delivery of chondrogenic factors such as TGFβ3 that promote chondrogenesis in situ or in vivo, circumventing laboratory cell manipulation and differentiation.

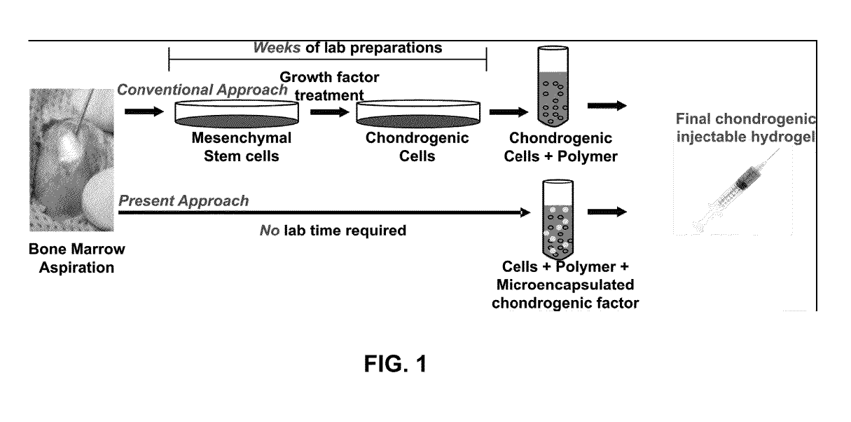

FIG. 2 is a series of images depicting chondrogenic tissue engineered construct preparation. FIG. 2A shows freshly harvested bone marrow plated in culture dish. FIG. 2B shows mesenchymal stem cells adhered to plastic culture dish for expansion in basic culture medium (no chondrogenic factors added). FIG. 2C shows Chitosan-GP in liquid form at room temperature and Chitosan-GP in gel form after 30 minutes at 37° C. (body temperature). FIG. 2D in an SEM image of PLGA microspheres encapsulating TGFβ3. FIG. 2E is a light microscopy image of Chitosan-GP gel mixed with hMSCs and TGFβ3 encapsulating PLGA microspheres (final injectable solution).

FIG. 3 is a series of images and a bar graph depicting chondrogenesis of bone marrow derived human mesenchymal stem cells (hMSC) in tissue engineered constructs. FIG. 3A shows chondrogenic differentiation of hMSC in monolayer culture supplemented with 10 ng/ml TGFβ3 demonstrating chondrogenic potential of hMSC. FIG. 3B shows tissue engineered thermosensitive chitosan construct containing TGFβ3 in PLGA microspheres seeded with hMSC. FIG. 3C shows DAPI staining of tissue engineered construct seeded with hMSC after 14 days of culture. FIG. 3D is a bar graph showing increase in Glycosaminoglycan (GAG) of hMSC cultured in thermosensitive chitosan with (right bar) or without (left bar) TGFβ3 loaded PLGA microspheres after 14 days. TGFβ3 from PLGA microspheres induced chondrogenesis in engineered constructs indicated by increased GAG content.

FIG. 4 is a bar graph showing matrix synthesis content of implants seeded with progenitor cells or 3D growth factor. The figure illustrates that controlled delivery of growth factor increases tissue synthesis in vivo. Matrix synthesis content of implants was determined after 4 weeks in subcutaneous pouch in the dorsum of immunodeficient mice. Controlled delivery of growth factor using PLGA microspheres without the delivery of any transplanted cells increased the amount of tissue synthesis compared to implants with transplanted MSCs. Further information regarding methodology is available in Example 5.

### DETAILED DESCRIPTION OF THE INVENTION

The approaches described herein are based, at least in part, upon application of the discovery that controlled release of TGFβ3 from microspheres can stimulate in situ chondrogenesis of untreated human mesenchymal stem cells in a biocompatible injectable matrix. Such observations provide for engineered constructs for the treatment of various tissue defects.

One aspect of the invention provides for an engineered construct that enables post-implantation differentiation of tissue progenitor cells. In various embodiments, the construct can contain controlled release growth factors. For example, in situ chondrogenesis by controlled delivery of TGFβ3 can significantly decrease fabrication time of chondrogenic constructs for the treatment of various conditions, such as cartilage degeneration. Tissue progenitor cells, such as bone marrow progenitors, can be readily isolated from patients and instantly mixed with the injectable controlled delivery gel system described herein. The autologous implant can then be injected back into the donor patient for tissue repair (e.g., cartilage repair) requiring minimal laboratory manipulation.

Generally, substantially undifferentiated and/or untreated tissue progenitor cells are introduced into the liquid phase of a multiphasic biocompatible matrix material, where they are stimulated to differentiate into the target tissue. Various growth factors can be supplied to the liquid matrix and/or the gelled matrix either directly or encapsulated in a controlled release delivery system.

Tissue Progenitor Cell

The compositions and methods of the invention generally employ tissue progenitor cells in seeding of a biocompatible matrix so as to form an engineered tissue construct. In various embodiments, the tissue progenitor cell is a precursor to tissue of interest and differentiates within the 3D matrix in the presence of growth factor in situ. Such cells can be isolated, purified, and/or cultured by a variety of means known to the art Methods for the isolation and culture of tissue progenitor cells are discussed in, for example, Vunjak-Novakovic and Freshney (2006) Culture of Cells for Tissue Engineering, Wiley-Liss, ISBN-10 0471629359. The tissue progenitor cells can be derived from the same or different species as the transplant recipient. For example, the progenitor cells can be derived from an animal, including, but not limited to, mammals, reptiles, and avians, more preferably horses, cows, dogs, cats, sheep, pigs, and chickens, and most preferably human. The tissue progenitor cells can be derived from the transplant recipient or from another subject of the same or different species.

Tissue progenitor cells infused into the matrix material are usually a progenitor cell capable of differentiating into, or otherwise forming, the target tissue or organ. For example, the tissue progenitor cell can be a mesenchymal stem cell (MSC), preferably a human MSC. MSCs are generally capable of differentiating into osteoblasts, chondrocytes, myocytes, adipocytes, neuronal cells, and beta-pancreatic islets cells, as well as other cells known in the art.

Preferably, the tissue progenitor cell is substantially undifferentiated. For example, the tissue progenitor cell can be freshly isolated and/or not pre-treated with growth factors before being introduced into the matrix.

Tissue progenitor cells can be present in the matrix at various amounts. Density-dependent inhibition of cell division (previously termed contact inhibition) can be a factor in cell survival, for example when mesenchymal stem cells give rise to osteogenic progenitor cells and end-stage osteoblasts in development (see Alberts et al., 2002). Too many cells seeded in an engineered tissue or organ scaffold can create shortage of locally available mitogens, growth factors and/or survival factors, potentially leading to apoptosis and causing unnecessary waste of in vitro cell expansion time (see Moioli and Mao, 2006). On the other hand, too few cells seeded in an engineered tissue or organ scaffold can lead to poor regeneration outcome. Various methodologies for optimizing the density of tissue progenitor cells so as to maximize the regenerative outcome of engineered vascularized tissue or organ are known to the art. Various matrix seeding densities can be monitored over time and at end-point cell densities with, for example, histology, structural analysis, immunohistochemistry, biochemical analysis, and mechanical properties. As will be recognized by one skilled in the art, the seeded cell densities of tissue progenitor cells can vary according to, for example, progenitor type, tissue or organ type, matrix material, matrix volume, infusion method, seeding pattern, culture medium, growth factors, incubation time, incubation conditions, and the like. Generally, the tissue progenitor cells can be present in the matrix material at a density of about 0.5 million cells (M) ml⁻¹ to about 100 M ml⁻¹. For example, the tissue progenitor cells and/or the vascular progenitor cells can be present in the matrix material at a density of about 1 M ml⁻¹, 5 M ml⁻¹, 10 M ml⁻¹, 15 M ml⁻¹, 20 M ml⁻¹, 25 M ml⁻¹, 30 M ml⁻¹, 35 M ml⁻¹, 40 M ml⁻¹, 45 M ml⁻¹, 50 M ml⁻¹, 55 M ml⁻¹, 60 M ml⁻¹, 65 M ml⁻¹, 70 M ml⁻¹, 75 M ml⁻¹, 80 M ml⁻¹, 85 M ml⁻¹, 90 M ml⁻¹, 95 M ml⁻¹, or 100 M ml⁻¹. As another example, the tissue progenitor cells and/or the vascular progenitor cells can be present in the matrix material at a density of about 1 M ml⁻¹ to about 5 M ml⁻¹, about 5 M ml⁻¹ to about 10 M ml⁻¹, about 10 M ml⁻¹ to about 15 M ml⁻¹, about 15 M ml⁻¹ to about 20 M ml⁻¹, about 20 M ml⁻¹ to about 25 M ml⁻¹, about 25 M ml⁻¹ to about 30 M ml⁻¹, about 30 M ml⁻¹ to about 35 M ml⁻¹, about 35 M ml⁻¹ to about 40 M ml⁻¹, about 40 M ml⁻¹ to about 45 M ml⁻¹, about 45 M ml⁻¹ to about 50 M ml⁻¹, about 50 M ml⁻¹ to about 55 M ml⁻¹, about 55 M ml⁻¹ to about 60 M ml⁻¹, about 60 M ml⁻¹ to about 65 M ml⁻¹, about 65 ml⁻¹ to about 70 M ml⁻¹, about 70 ml⁻¹ to about 75 M ml⁻¹, about 75 ml⁻¹ to about 80 M ml⁻¹, about 80 ml⁻¹ to about 85 M ml⁻¹, about 85 ml⁻¹ to about 90 M ml⁻¹, about 90 ml⁻¹ to about 95 M ml⁻¹, or about 95 ml⁻¹ to about 100 M ml⁻¹.

In some embodiments, the progenitor cells used to seed the matrix are transformed with a heterologous nucleic acid so as to express a bioactive molecule, or heterologous protein or to overexpress an endogenous protein. As an example, the progenitor cells to be seeded in the matrix can be genetically modified to expresses a fluorescent protein marker. Exemplary markers include GFP, EGFP, BFP, CFP, YFP, and RFP. As another example, progenitor cells to be seeded in the matrix can be genetically modified to express an angiogenesis-related factor, such as activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin and fibronectin receptor α5β1, Factor X, HB-EGF, HBNF, HGF, HUAF, heart derived inhibitor of vascular cell proliferation, IFN-gamma, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-beta, Tie 1, Tie2, TGF-β, and TGF-β receptors, TIMPs, TNF-alpha, TNF-beta, transferrin, thrombospondin, urokinase, VEGF-A, VEGF-B, VEGF-C, VEGF-D, VEGF-E, VEGF, VEGF.sub.164, VEGI, EG-VEGF, VEGF receptors, PF4, 16 kDa fragment of prolactin, prostaglandins E1 and E2, steroids, heparin, 1-butyryl glycerol (monobutyrin), and/or nicotinic amide. As another example, progenitor cells to be seeded in the matrix can be transfected with genetic sequences that are capable of reducing or eliminating an immune response in the host (e.g., expression of cell surface antigens such as class I and class II histocompatibility antigens can be suppressed). This can allow the transplanted cells to have reduced chance of rejection by the host.

In some embodiments, the matrix material can be seeded with one or more cell types in addition to the first tissue progenitor cell. Such additional cell type can be selected from those discussed above, and/or can include (but are not limited to) skin cells, liver cells, heart cells, kidney cells, pancreatic cells, lung cells, bladder cells, stomach cells, intestinal cells, cells of the urogenital tract, breast cells, skeletal muscle cells, skin cells, bone cells, cartilage cells, keratinocytes, hepatocytes, gastro-intestinal cells, epithelial cells, endothelial cells, mammary cells, skeletal muscle cells, smooth muscle cells, parenchymal cells, osteoclasts, and/or chondrocytes. These cell-types can be introduced prior to, during, or after implantation of the seeded matrix. Such introduction can take place in vitro and/or in vivo. When the cells are introduced in vivo, the introduction can be at the site of the engineered vascularized tissue or organ composition and/or at a site removed therefrom. Exemplary routes of administration of the cells include injection and surgical implantation.

Bioactive Molecules

One or more bioactive molecules can be introduced into the matrix so as to, inter alia, induce stem cell differentiation. For example, the bioactive molecule can be a growth factor, preferably a tissue growth factor, more preferably TGFβ3. Such a growth factor can be supplied at, for example, a concentration of about 0 to 1000 ng/mL. For example, the growth factor can be present at a concentration of about 100 to 700 ng/mL, at a concentration of about 200 to 400 ng/mL, or at a concentration of about 250 ng/mL. As another example, the growth factor can be present at a concentration of about 100 ng/mL, about 150 ng/mL, about 200 ng/mL, about 250 ng/mL, about 300 ng/mL, about 400 ng/mL, about 450 ng/mL, about 500 ng/mL, about 550 ng/mL, about 600 ng/mL, about 650 ng/mL, or about 700 ng/mL.

The cells of the matrix can be, for example, genetically engineered to express the bioactive molecule or the bioactive molecule can be added to the matrix. The matrix can also be cultured in the presence of the bioactive molecule. The bioactive molecule can be added prior to, during, or after seeding the matrix with the progenitor cells. Non-limiting examples of bioactive molecules include activin A, adrenomedullin, aFGF, ALK1, ALK5, ANF, angiogenin, angiopoietin-1, angiopoietin-2, angiopoietin-3, angiopoietin-4, angiostatin, angiotropin, angiotensin-2, AtT20-ECGF, betacellulin, bFGF, B61, bFGF inducing activity, cadherins, CAM-RF, cGMP analogs, ChDI, CLAF, claudins, collagen, collagen receptors α₁β₁ and α₂β₁, connexins, Cox-2, ECDGF (endothelial cell-derived growth factor), ECG, ECI, EDM, EGF, EMAP, endoglin, endothelins, endostatin, endothelial cell growth inhibitor, endothelial cell-viability maintaining factor, endothelial differentiation shpingolipid G-protein coupled receptor-1 (EDG1), ephrins, Epo, HGF, TNF-alpha, TGF-beta, PD-ECGF, PDGF, IGF, IL8, growth hormone, fibrin fragment E, FGF-5, fibronectin, fibronectin receptor α₅β₁, Factor X, HB-EGF, HBNF, HGF, HUAF, heart derived inhibitor of vascular cell proliferation, IFN-gamma, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-β, PD-ECGF, PAI-2, PD-ECGF, PF4, P1 GF, PKR1, PKR2, PPAR.gamma., PPARγ ligands, phosphodiesterase, prolactin, prostacyclin, protein S, smooth muscle cell-derived growth factor, smooth muscle cell-derived migration factor, sphingosine-1-phosphate-1 (S1P1), Syk, SLP76, tachykinins, TGF-β, Tie 1, Tie2, TGF-βreceptors, TIMPs, TNF-alpha, TNF-beta, transferrin, thrombospondin, urokinase, VEGF-A, VEGF-B, VEGF-C, VEGF-D, VEGF-E, VEGF, VEGF₁₆₄, VEGI, EG-VEGF, VEGF receptors, PF4, 16 kDa fragment of prolactin, prostaglandins E1 and E2, steroids, heparin, 1-butyryl glycerol (monobutyrin), and/or nicotinic amide. In other preferred embodiments, the matrix can include a chemotherapeutic agent or immunomodulatory molecule. Such agents and molecules are known to the skilled artisan.

Biologic drugs that can be added to the compositions of the invention include immunomodulators and other biological response modifiers. A biological response modifier generally encompasses a biomolecule (e.g., peptide, peptide fragment, polysaccharide, lipid, antibody) that is involved in modifying a biological response, such as the immune response or tissue or organ growth and repair, in a manner that enhances a particular desired therapeutic effect, for example, the cytolysis of bacterial cells or the growth of tissue- or organ-specific cells or vascularization. Biologic drugs can also be incorporated directly into the matrix component. Those of skill in the art will know, or can readily ascertain, other substances which can act as suitable non-biologic and biologic drugs.

Compositions of the invention can also be modified to incorporate a diagnostic agent, such as a radiopaque agent. The presence of such agents can allow the physician to monitor the progression of wound healing occurring internally. Such compounds include barium sulfate as well as various organic compounds containing iodine. Examples of these latter compounds include iocetamic acid, iodipamide, iodoxamate meglumine, iopanoic acid, as well as diatrizoate derivatives, such as diatrizoate sodium. Other contrast agents that can be utilized in the compositions of the invention can be readily ascertained by those of skill in the art and can include, for example, the use of radiolabeled fatty acids or analogs thereof.

The concentration of agent in the composition will vary with the nature of the compound, its physiological role, and desired therapeutic or diagnostic effect. A therapeutically effective amount is generally a sufficient concentration of therapeutic agent to display the desired effect without undue toxicity. A diagnostically effective amount is generally a concentration of diagnostic agent which is effective in allowing the monitoring of the integration of the tissue graft, while minimizing potential toxicity. In any event, the desired concentration in a particular instance for a particular compound is readily ascertainable by one of skill in the art.

The matrix composition can be enhanced, or strengthened, through the use of supplements, such as human serum albumin (HSA), hydroxyethyl starch, dextran, or combinations thereof. The solubility of the matrix compositions can also be enhanced by the addition of a nondenaturing nonionic detergent, such as polysorbate 80. Suitable concentrations of these compounds for use in the compositions of the invention will be known to those of skill in the art, or can be readily ascertained without undue experimentation. The matrix compositions can also be further enhanced by the use of optional stabilizers or diluents. The proper use of these would be known to one of skill in the art, or can be readily ascertained without undue experimentation.

Encapsulation

The growth factors for use in the present invention can be encapsulated within a polymeric delivery systems so as to provide for controlled release of tissue growth factor from within the matrix. For example, the polymeric delivery system can be a polymeric microsphere, preferably a PLGA polymeric microspheres. A variety of polymeric delivery systems, as well as methods for encapsulating a molecule such as a growth factor, are known to the art (see e.g., Varde and Pack (2004) Expert Opin Biol Ther 4, 35-51).

Matrix

The compositions and methods of the invention employ a matrix, into or onto which undifferentiated tissue progenitor cells can be seeded. Such matrix materials can: allow cell attachment and migration; deliver and retain cells and biochemical factors; enable diffusion of cell nutrients and expressed products; and/or exert certain mechanical and biological influences to modify the behavior of the cell phase. The matrix is generally a porous, microcellular scaffold of a biocompatible material that provides a physical support and an adhesive substrate for seeding vascular progenitor cells and tissue progenitor cells during in vitro culturing and subsequent in vivo implantation.

A matrix with different phases of viscosity is preferred. For example, a matrix that can have a substantially liquid phase and a substantially gelled phase is preferred. The transition between phases can be stimulated by a variety of factors including, but limited to, light, chemical, magnetic, electrical, and mechanical stimulus. For example, the matrix can be a thermosensitive matrix with a substantially liquid phase at about room temperature and a substantially gelled phase at about body temperature. Room temperature is generally understood to denote a temperature range of about 15° C. to about 25° C., more preferably about 18° C. to about 23° C. In various configurations, a temperature-sensitive biphasic matrix material can make a transition from a substantially liquid phase to a gelled phase at any temperature from just above room temperature to about the temperature of a mammalian body (e.g., about 37° C. in a human), so long as the biphasic matrix material gels upon introduction to a mammalian body. As such, when it is said that a biphasic matrix material has a gelled phase at about 37° C., it does not necessarily mean that about 37° C. is the lowest temperature at which the biphasic matrix material makes a transition between liquid and gel phase; but rather, that upon temperature elevation to about 37° C. (e.g., via injection into a human body), the biphasic matrix material will transition between liquid and gel phase. Preferably, the liquid phase of the matrix has a lower viscosity that provides for optimal distribution of cells and injectability, while the solid phase of the matrix has an elevated viscosity that provides for seeded matrix retention at or within the target tissue.

The solid phase of the matrix should have an adequate porosity and an adequate pore size so as to facilitate cell seeding and diffusion throughout the whole structure of both cells and nutrients. Matrix biodegradability is also preferred since absorption of the matrix by the surrounding tissues can eliminate the necessity of a surgical removal. The rate at which degradation occurs should coincide as much as possible with the rate of tissue or organ formation. Thus, while cells are fabricating their own natural structure around themselves, the matrix is able to provide structural integrity and eventually break down, leaving the neotissue, newly formed tissue or organ which can assume the mechanical load. Injectability is also preferred in some clinical applications. Suitable matrix materials are discussed in, for example, Ma and Elisseeff, ed. (2005) Scaffolding In Tissue Engineering, CRC, ISBN 1574445219; Saltzman (2004) Tissue Engineering: Engineering Principles for the Design of Replacement Organs and Tissues, Oxford ISBN 019514130X. As an example, a suitable matrix material for use in the present invention is chitosan—glycerol 2-phosphate disodium salt hydrate (GP) liquid/gel, a thermosensitive, injectable, biocompatible matrix.

The matrix can be formed of synthetic polymers. Such synthetic polymers include, but are not limited to, polyurethanes, polyorthoesters, polyvinyl alcohol, polyamides, polycarbonates, polyvinyl pyrrolidone, marine adhesive proteins, cyanoacrylates, analogs, mixtures, combinations and derivatives of the above. Alternatively, the matrix can be formed of naturally occurring biopolymers. Such naturally occurring biopolymers include, but are not limited to, fibrin, fibrinogen, fibronectin, collagen, and other suitable biopolymers. Also, the matrix can be formed from a mixture of naturally occurring biopolymers and synthetic polymers.

The matrix material the matrix can include, for example, a collagen gel, a polyvinyl alcohol sponge, a poly(D,L-lactide-co-glycolide) fiber matrix, a polyglactin fiber, a calcium alginate gel, a polyglycolic acid mesh, polyester (e.g., poly-(L-lactic acid) or a polyanhydride), a polysaccharide (e.g. alginate), polyphosphazene, polyacrylate, and/or a polyethylene oxide-polypropylene glycol block copolymer. Matrices can be produced from proteins (e.g. extracellular matrix proteins such as fibrin, collagen, and fibronectin), polymers (e.g., polyvinylpyrrolidone), or hyaluronic acid. Synthetic polymers can also be used, including bioerodible polymers (e.g., poly(lactide), poly(glycolic acid), poly(lactide-co-glycolide), poly(caprolactone), polycarbonates, polyamides, polyanhydrides, polyamino acids, polyortho esters, polyacetals, polycyanoacrylates), degradable polyurethanes, non-erodible polymers (e.g., polyacrylates, ethylene-vinyl acetate polymers and other acyl substituted cellulose acetates and derivatives thereof), non-erodible polyurethanes, polystyrenes, polyvinyl chloride, polyvinyl fluoride, poly(vinylimidazole), chlorosulphonated polyolifins, polyethylene oxide, polyvinyl alcohol, teflon®, and/or nylon.

To form the compositions of various configurations of the invention, tissue progenitor cells can be introduced (e.g., implanted, infused, or seeded) into the substantially liquid phase of a matrix capable of forming a gelled phase matrix. The tissue progenitor cells can be introduced in a homogenous or heterogenous distribution throughout the liquid. It is contemplated that more than one type of tissue progenitor cell can be introduced into the matrix.

Implanting

The engineered tissue or organ compositions of the invention hold significant clinical value because of their ability to be formed from substantially undifferentiated tissue progenitor cells without the need for 2D culturing, pretreatment, and/or pre-differentiation. A determination of the need for treatment will typically be assessed by a history and physical exam consistent with the tissue or organ defect at issue. Subjects with an identified need of therapy include those with a diagnosed tissue or organ defect. The subject is preferably an animal, including, but not limited to, mammals, reptiles, and avians, more preferably horses, cows, dogs, cats, sheep, pigs, and chickens, and most preferably human.

As an example, a subject in need can have a deficiency of at least about 5%, about 10%, about 25%, about 50%, about 75%, about 90% or more of a particular cell type. As another example, a subject in need can have damage to a tissue or organ, and the method provides an increase in biological function of the tissue or organ by at least about 5%, about 10%, about 25%, about 50%, about 75%, about 90%, about 100%, or about 200%, or even by as much as about 300%, about 400%, or about 500%. As yet another example, the subject in need can have a disease, disorder, or condition, and the method provides an engineered tissue or organ construct sufficient to ameliorate or stabilize the disease, disorder, or condition. For example, the subject can have a disease, disorder, or condition that results in the loss, atrophy, dysfunction, and/or death of cells. Exemplary treated conditions include a neural, glial, or muscle degenerative disorder, muscular atrophy or dystrophy, heart disease such as congenital heart failure, hepatitis or cirrhosis of the liver, an autoimmune disorder, diabetes, cancer, a congenital defect that results in the absence of a tissue or organ, or a disease, disorder, or condition that requires the removal of a tissue or organ, ischemic diseases such as angina pectoris, myocardial infarction and ischemic limb, and/or accidental tissue defect or damage such as fracture or wound. In a further example, the subject in need can have an increased risk of developing a disease, disorder, or condition that is delayed or prevented by the method.

The tissue or organ can be selected from bladder, brain, nervous tissue, glia, esophagus, fallopian tube, heart, pancreas, intestines, gall bladder, kidney, liver, lung, ovaries, prostate, spinal cord, spleen, stomach, testes, thymus, thyroid, trachea, urogenital tract, ureter, urethra, uterus, breast, skeletal muscle, skin, adipose, bone, and cartilage. The vascular progenitor cells and/or tissue progenitors cells can be from the same subject into which the engineered tissue composition is grafted. Alternatively, the progenitor cells can be from the same species, or even different species.

Implantation of an engineered tissue or organ construct is within the skill of the art. Preferably, a tissue progenitor cell seeded matrix is injected in a substantially liquid phase, and upon or soon after introduction to the target tissue, the matrix polymerizes or is induced to polymerize so as to be retained in the tissue.

The matrix and cellular assembly can be either fully or partially implanted into a tissue or organ of the subject to become a functioning part thereof. Preferably, the implant initially attaches to and communicates with the host through a cellular monolayer. Over time, the seeded cells can expand and migrate out of the polymeric matrix to the surrounding tissue. After implantation, cells surrounding the engineered vascularized tissue composition can enter through cell migration. The cells surrounding the engineered tissue can be attracted by biologically active materials, including biological response modifiers, such as polysaccharides, proteins, peptides, genes, antigens, and antibodies, which can be selectively incorporated into the matrix to provide the needed selectivity, for example, to tether the cell receptors to the matrix, stimulate cell migration into the matrix, or both. Generally, the gelled phase of the matrix is porous, having interconnecting channels that allow for cell migration, augmented by both biological and physical-chemical gradients. For example, cells surrounding the implanted seeded matrix can be attracted by biologically active materials including one or more of VEGF, fibroblast growth factor, transforming growth factor-beta, endothelial cell growth factor, P-selectin, and intercellular adhesion molecule. One of skill in the art will recognize and know how to use other biologically active materials that are appropriate for attracting cells to the matrix.

The methods, compositions, and devices of the invention can include concurrent or sequential treatment with one or more of enzymes, ions, growth factors, and biologic agents, such as thrombin and calcium, or combinations thereof. The methods, compositions, and devices of the invention can include concurrent or sequential treatment with non-biologic and/or biologic drugs.

Having described the invention in detail, it will be apparent that modifications, variations, and equivalent embodiments are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention. It shall be understood that any method described in an example may or may not have been actually performed, or any composition described in an example may or may not have been actually been formed, regardless of verb tense used.

### Example 1

### Thermosensitive Chitosan Gel Fabrication

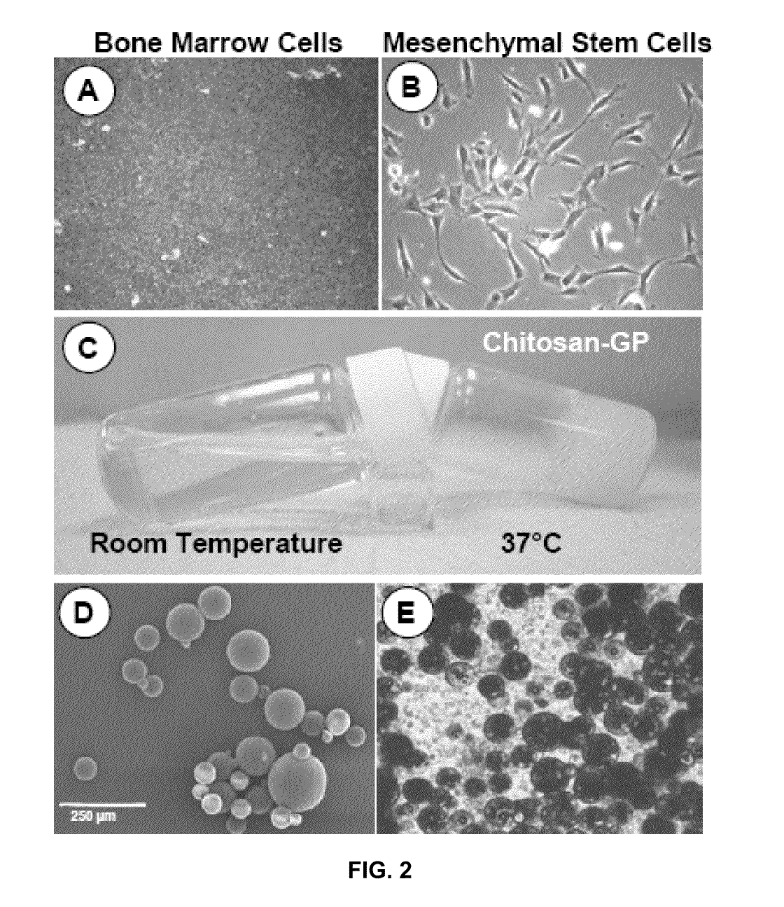

Chitosan (source: crustacean chitin; ICN Biomedicals Inc., Aurora, Ohio) was dissolved in 1N HCl for 24 hrs and autoclaved at 121° C. for 10 mins. Filter sterile 55% glycerol 2-phosphate disodium salt hydrate (GP) (FW:216.04; Sigma, St. Louis, Mo.) was added to chitosan solution on ice bath to form the thermosensitive gel (2% chitosan, 8.2% GP). This solution is in liquid form at RT and gels at 37° C. (FIG. 2). Previous studies on the optimization of sterilization procedures of chitosan revealed that autoclave for 10 minutes at 121° C. maintains functionality of fabricated gels [12]. Sterilization of each solution was performed separately since temperature rise would gel the mixed solution.

### Example 2

### Fabrication of PLGA Microspheres Encapsulating TGFβ3

Microspheres of poly(DL-lactic-co-glycolic acid) (PLGA; Sigma, St. Louis, Mo.) of 50:50 PLA:PGA ratio (Sigma, St. Louis, Mo.) were prepared using double emulsion technique ((water-in-oil)-in-water) (FIGS. 2D, 2E). A total of 250 mg PLGA was dissolved into 1 ml dichloromethane. Recombinant human TGFβ3 with molecular weight of 25 kDa (R&D Systems, Minneapolis, Minn.) was diluted in 50 μl of reconstituting solution per manufacturer protocol and added to the PLGA solution, forming a mixture (primary emulsion) that was emulsified for 1 min (water-in-oil). The primary emulsion was then added to 2 ml of 1% polyvinyl alcohol (PVA, MW 30,000-70,000), followed by 1 min mixing ((water-in-oil)-in-water).

Upon adding 100 ml 0.1% PVA solution, the mixture was stirred for 1 min. A total of 100 ml of 2% isopropanol was added to the final emulsion and continuously stirred for 2 hrs under chemical hood to remove the solvent. PLGA microspheres containing TGFβ3 were isolated using filtration (2 μm filter) and washed with distilled water. Microspheres were frozen in liquid nitrogen for 30 min and lyophilized for 48 hrs. Freeze-dried PLGA microspheres were stored at −20° C. prior to use.

### Example 3

### Construct Formation and Controlled Release of TGFβ3

Human mesenchymal stem cells (hMSCs) were seeded in thermosensitive chitosan gels at 5×10⁶ cells/ml at room temperature for all experimental groups. PLGA microspheres encapsulating TGFβ3 (as formed in Example 2) were added to cell/chitosan solution (as formed in Example 1) to sustain continuous release of approximately 10 ng/ml TGFβ3. Cell/Chitosan/Microspheres construct was injected into the wells of a 96-well plate at room temperature and allowed to gel at 37° C. in incubator.

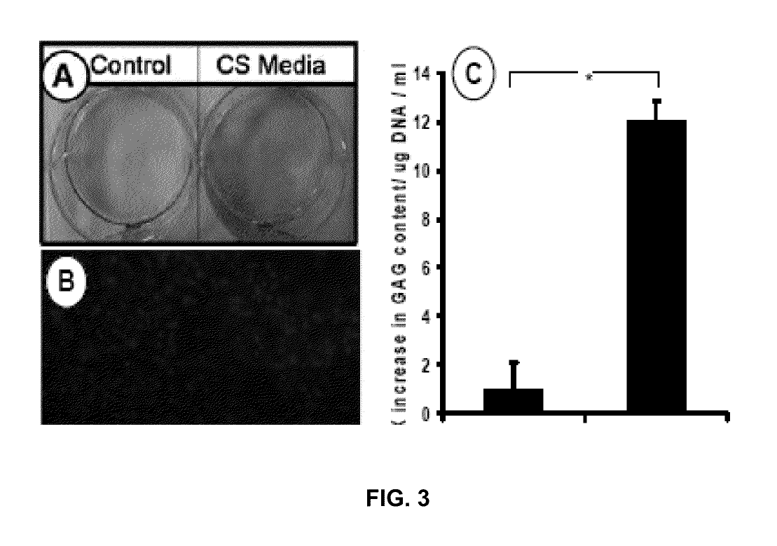

Results showed that TGFβ3 was released from PLGA microspheres within the tissue engineered constructs. TGFβ3 was released in a sustained fashion up to 4 weeks from PLGA microspheres embedded in chitosan-GP thermosensitive gels. The typical initial burst release over the first few days (4-7 days) was not observed for the case of chitosan-GP embedded PLGA microspheres (see e.g., FIG. 3). The R² value representing linear correlation in the release curve was 0.99, which characterizes high linearity.

### Example 4

### Chondrogenic Effects of TGFβ3

The chondrogenic effects of TGFβ3 on hMSCs were verified using a monolayer culture system. Human MSCs were plated in 6-well plates at high densities (2.5×10⁶ cells/ml) and cultured under chondrogenic supplements including 10 ng/ml of TGFβ3.

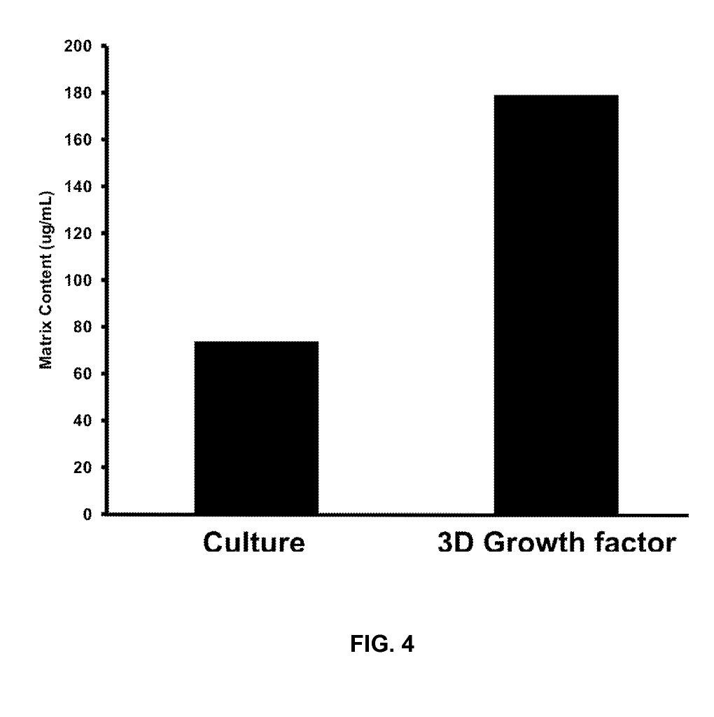

Results showed that hMSCs differentiated into chondrocyte-like cells that produced cartilage-like matrix after 14 days in culture as suggested by alcian blue staining of sulfated glycosaminoglycans, a typical cartilage ECM molecule (see e.g., FIG. 4A).

### Example 5

### In Situ Chondrogenesis

In situ chondrogenesis within the constructs described above were examined. Human MSCs were isolated from the bone marrow of healthy donors by negative selection and culture plated for expansion using growth medium with no chondrogenic factors added. Cell/chitosan/TGFβ3 microsphere constructs were formed as described in Example 3. As control, cell/chitosan constructs were formed without encapsulated TGFβ3.

Results showed that in situ chondrogenesis of hMSCs occurred in thermosensitive chitosan-GP gels during controlled delivery of TGFβ3. Without any prior chondrogenic treatment, hMSCs seeded in chitosan-GP gels that included controlled delivery of TGFβ3 by PLGA microspheres (see e.g., FIG. 4B) exhibited chondrogenic differentiation and synthesis of cartilage-like extracellular matrix molecules such as glycosaminoglycans (GAG) after 10 days of culture (see e.g., FIG. 4D). Controls, which included hMSCs seeded at the same density in chitosan-GP gels, without TGFβ3, had significantly lower GAG content. The observed increase in GAG production by hMSCs cultured with controlled delivery of TGFβ3 within the chondrogenic injectable constructs was approximately 12-fold. Cell seeding was confirmed by DAPI nuclear staining of gels (see e.g., FIG. 4C).

These findings suggest that the laborious pre-differentiation of bone marrow derived stem cells, which can require many weeks of cell manipulations and growth factor treatment, can be circumvented by the present approach.

What is claimed is:

1. An engineered tissue composition comprising:
a substantially undifferentiated tissue progenitor cell;
a tissue growth factor or a tissue growth factor encapsulated in a controlled release delivery system; and
a biphasic matrix material having a substantially liquid phase and a gelled phase;
wherein the substantially undifferentiated tissue progenitor cell and the tissue growth factor or the encapsulated tissue growth factor are infused in the substantially liquid phase biphasic matrix material.

2. A method of forming an engineered tissue composition comprising:
providing a substantially undifferentiated tissue progenitor cell; a tissue growth factor or a tissue growth factor encapsulated in a controlled release delivery system; and a biphasic matrix material having a substantially liquid phase and a gelled phase;
introducing the substantially undifferentiated tissue progenitor cell into the substantially liquid phase of the biphasic matrix material;
introducing the tissue growth factor or the encapsulated tissue growth factor into the substantially liquid phase of the biphasic matrix material;
forming a substantially liquid phase tissue composition comprising the biphasic matrix material, the tissue progenitor cell, and the tissue growth factor or the encapsulated tissue growth factor.

3. The method of claim 2 further comprising the step of forming a gelled tissue composition from the substantially liquid phase tissue composition.

4. (canceled)

5. A method of treating a tissue defect comprising:
(a) introducing the composition of claim 1 into a subject in need thereof; or
(b) (i) providing a substantially undifferentiated tissue progenitor cell; a tissue growth factor or a tissue growth factor encapsulated in a controlled release delivery system; and a biphasic matrix material having a substantially liquid phase and a gelled phase;
(ii) introducing the substantially undifferentiated tissue progenitor cell into the substantially liquid phase of the biphasic matrix material;
(iii) introducing the tissue growth factor or the encapsulated tissue growth factor into the substantially liquid phase of the biphasic matrix material; and
(iv) introducing a composition comprising the biphasic matrix material, the tissue progenitor cell, and the tissue growth factor or the encapsulated tissue growth factor into a subject in need thereof.

6. The method of claim 5 wherein at least a portion of the tissue progenitor cells remain substantially undifferentiated upon introduction of the composition into the subject.

7. The method of claim 5 wherein a majority of the tissue progenitor cells remain substantially undifferentiated upon introduction of the composition into the subject.

8. The method of claim 5 wherein substantially all of the tissue progenitor cells remain substantially undifferentiated upon introduction of the composition into the subject.

9. The method of 5 wherein the substantially undifferentiated tissue progenitor cells differentiate in situ after introduction of the composition into the subject.

10. The method of 5 wherein the subject is a mammalian subject.

11. (canceled)

12. The method of claim 10 wherein the mammalian subject is a human subject.

13. The composition of claim 1 wherein the biphasic matrix material is a thermosensitive biphasic matrix material having a substantially liquid phase at about room temperature and a gelled phase at about 37° C.

14. The composition of claim 1 wherein the biphasic matrix material is injectable in the substantially liquid phase and is in the gelled phase after injection into a subject.

15. The method of claim 14 wherein introducing the composition to the subject comprises injecting the liquid phase composition into the subject.

16. The composition of claim 1 wherein the tissue progenitor cell is a mesenchymal stem cell (MSC).

17. The composition of claim 16 wherein the MSC is a human mesenchymal stem cell (hMSC).

18. The composition of claim 1 wherein the tissue growth factor is a chondrogenic growth factor.

19. The composition of claim 1 wherein the tissue growth factor is TGFβ3.

20. The composition of claim 1 wherein the controlled release delivery system is a polymeric microsphere.

21. The composition of claim 20 wherein the polymeric microsphere is a poly(DL-lactic-co-glycolic acid) (PLGA) microsphere.

22. The composition of claim 1 wherein the biphasic matrix material is a chitosan-GP matrix.

23. The composition of claim 1 wherein the substantially undifferentiated tissue progenitor cell is not treated with a growth factor before being introduced into the biphasic matrix material.
